(19) United States
(12) Patent Application Publication
(10) Pub. No.: US 2025/0076237 A1
(43) Pub. Date: Mar. 6, 2025

(54) THERMAL RUNAWAY DETECTION AND CONTROL DEVICE AND METHOD THEREOF

(71) Applicant: INDUSTRY-ACADEMIC COOPERATION FOUNDATION, YONSEI UNIVERSITY, Seoul (KR)

(72) Inventors: Woo Young LEE, Seoul (KR); Kyu Sik SHIN, Seoul (KR)

(21) Appl. No.: 18/817,640

(22) Filed: Aug. 28, 2024

(30) Foreign Application Priority Data
Aug. 28, 2023 (KR) 10-2023-0112777
Aug. 28, 2023 (KR) 10-2023-0112783
Aug. 28, 2023 (KR) 10-2023-0112786
May 23, 2024 (KR) 10-2024-0067089

(51) Int. Cl. G01N 27/12; B82Y 15/00; G01N 33/00; H01M 10/42; H01M 10/44

(52) U.S. Cl. CPC G01N 27/127; B82Y 15/00; H01M 10/425; H01M 10/44; G01N 33/005; H01M 2010/4271

(57) ABSTRACT

The present disclosure relates to a thermal runaway of lithium-ion battery detection and control device including: sensor modules for detecting and monitoring hydrogen concentrations of battery cells and each having a palladium-based nano sensor and a communication unit; and a battery cell controller for turning on and off charging/discharging switches of the battery cells according to the hydrogen concentrations detected through the sensor modules and variations in the hydrogen concentrations according to time, wherein the palladium-based nano sensor detects the hydrogen concentration between 20 and 400 ppm.

### BACKGROUND OF THE DISCLOSURE

### Cross Reference to Related Application of the Disclosure

The present application claims the benefit of Korean Patent Application No. 10-2023-0112777 filed in the Korean Intellectual Property Office on August 28, 2023, No. 10-2023-0112783 filed in the Korean Intellectual Property Office on August 28, 2023, No. 10-2023-0112786 filed in the Korean Intellectual Property Office on August 28, 2023 and No. 10-2024-0067089 filed in the Korean Intellectual Property Office on May 23, 2024, the entire contents of which are incorporated herein by reference.

### FIELD OF THE DISCLOSURE

The present disclosure relates to a thermal runaway detection and control device and method, more specifically to a thermal runaway detection and control device and method that is capable of mounting palladium-based nano sensors on individual cells of a lithium-ion battery of an energy storage system (ESS) to monitor hydrogen concentrations and controlling charging/discharging switches of the individual cells, thereby pre-sensing thermal runaway of the lithium-ion battery to prevent the thermal runaway from occurring.

### BACKGROUND OF THE RELATED ART

Battery thermal runaway occurs due to high temperature, overcharging, physical damage, or chemical malfunction, which causes a serious problem. Such a problem happens by various reasons, such as lack of understanding about the inside structure or chemical reaction of a battery, a defect in a battery manufacturing process, the use of a charger not fit for purpose, and the like.

FIG. 1 is a flowchart showing a process of causing thermal runaway in a lithium-ion battery.

At step S10, battery abuse as one of reasons of thermal runaway occurs. In this case, the battery abuse includes high temperature exposure, overcharging, physical damage, chemical malfunction, etc.

For example, if the battery is exposed to a high temperature for a long period of time, the inside structure of the battery is damaged and a chemical reaction speed of the battery increases to generate heat from the battery.

Further, if the battery is overcharged, an internal chemical reaction is excessively activated to generate heat from the battery. This happens when a charger is malfunctioned or an electric current over battery capacity flows.

Furthermore, the physical damage represents the internal structure (e.g., separation film) of the battery is damaged due to physical damage such as an impact or pressure to cause chemical substances to be exposed so that chemical reactions increase and heat is generated.

Besides, the chemical malfunction happens when chemical heat is generated due to a trouble in a chemical process inside the battery. This generally happens due to malfunctions or corrosion of the components of the battery.

If irresistible physical damage is not applied from the outside to the battery, the battery abuse as the thermal runaway happens generally in the process of being charged and discharged.

At step S20 as the initial step of the thermal runaway due to the battery abuse, a battery temperature rises, and gases start to be produced from the inside of the battery through a chemical reaction. In detail, thermal instability in the inner materials of the battery is caused, an electrolyte starts to be dissolved, and the dissolved material is produced to the form of gases. As the gases are produced, the internal pressure of the battery increases.

A safety device built in the battery operates due to the increasing internal pressure, thereby causing primary venting. At step S30, off-gases having various chemical substances such as hydrogen (H₂), carbon dioxide (CO₂), carbon monoxide (CO), hydrogen fluoride (HF), hydrogen chloride (HCl), sulfur dioxide (SO₂), and the like are emitted.

In this case, the off-gas initially emitted is hydrogen, and at an initial step, hydrogen is emitted in concentration of 20 to 200 ppm at a temperature of 35° C. or above.

At step S40, after that, thermal runway happens so that the battery temperature rapidly rises and additional heat is generated from the interior of the battery due to an internal short circuit. As a result, smoke is generated due to secondary venting.

At step S50, battery fire and explosion occur, which may be applied to neighboring battery cells. As a result, the thermal runaway may cause big scale fire or explosion.

To prevent the battery thermal runaway from occurring, various technical solutions have been suggested. For example, there are a method for optimizing the internal structure of a battery in a step of designing the battery to improve heat dissipation, a method for sensing and controlling overcharging through a battery management system (BMS), and a method for preventing physical damage from occurring using a battery case and a protection device that have good durability.

Further, development in sensor technologies for quickly sensing and controlling heat generated from the battery has an important role in preventing thermal runaway from occurring. Such sensor technologies are consistently studied and developed to ensure safe use of batteries.

However, the conventional technologies just serve to sense gases for the whole of ESS or battery pack consisting of a plurality of battery cells, thereby having a limitation in sensing a small amount of gas for the respective battery cells.

Therefore, there is a need to develop a technology that is capable of sensing a small amount of hydrogen gas first produced among off-gases and controlling a battery using the sensed result, thereby efficiently preventing thermal runaway from occurring.

### SUMMARY OF THE DISCLOSURE

Accordingly, the present disclosure has been made in view of the above-mentioned problems occurring in the related art, and it is an object of the present disclosure to provide a thermal runaway detection and control device and method for an energy storage system that is capable of quickly detecting thermal runaway using palladium-based nano sensors mounted onto individual battery cells.

It is another object of the present disclosure to provide a thermal runaway detection and control device and method for an energy storage system that is capable of detecting a low concentration of hydrogen gas produced at an initial step of thermal runaway and controlling a charging/discharging switch of an individual battery cell according to variations in the hydrogen concentration detected.

It is yet another object of the present disclosure to provide a thermal runaway detection and control device and method for an energy storage system that is capable of controlling a charging/discharging switch according to stable or unstable state of an individual battery cell, after the charging/discharging switch of the corresponding individual battery cell has been turned off to control thermal runaway, thereby improving the efficiency in use of the battery cell.

To accomplish the above-mentioned objects, according to one aspect of the present disclosure, there is provided a thermal runaway detection and control device including: sensor modules for detecting and monitoring hydrogen concentrations of battery cells and each having a palladium-based nano sensor and a communication unit; and a battery cell controller for turning on and off charging/discharging switches of the battery cells according to the hydrogen concentrations detected through the sensor modules and variations in the hydrogen concentrations according to time, wherein the palladium-based nano sensor detects the hydrogen concentration between 20 and 400 ppm.

According to the present disclosure, desirably, the palladium-based nano sensor may include at least one of a palladium-based nanogap sensor and a palladium-based nanorod sensor, and the palladium-based nanogap sensor and the palladium-based nanorod sensor may be switched from an off state to an on state according to the hydrogen concentration detected.

According to the present disclosure, desirably, the palladium-based nano sensor may include a plurality of palladium-based nanogap sensors or palladium-based nanorod sensors having different detection ranges.

According to the present disclosure, desirably, the battery cell controller may include: a hydrogen concentration receiving unit for receiving the hydrogen concentrations from the sensor modules; a hydrogen concentration monitoring unit for monitoring the hydrogen concentrations in real time; a battery cell state determining unit for determining the states of the battery cells, based on the results monitored in real time through the hydrogen concentration monitoring unit; and a cell charging/discharging switch for turning on and off the charging/discharging switches of the battery cells according to the determined results of the battery cell state determining unit.

According to the present disclosure, desirably, the hydrogen concentration monitoring unit may monitor a first reference value in the hydrogen concentration through which it is determined that the corresponding battery cell is in an unstable state and a second reference value in the hydrogen concentration through which it is determined that the corresponding battery cell is in a risky state.

According to the present disclosure, desirably, the first reference value may be determined between 20 and 50 ppm, and the second reference value may be determined between 200 and 400 ppm.

According to the present disclosure, desirably, the battery cell state determining unit may determine the states of the battery cells as stable, unstable, and risky states according to non-detection of hydrogen, the hydrogen concentration reaching the first reference value, and the hydrogen concentration reaching the second reference value.

According to the present disclosure, desirably, if the hydrogen concentration of the corresponding battery cell reaches the second reference value 75 seconds before from the detection of hydrogen production, the battery cell state determining unit may determine the state of the corresponding battery cell as the risky state and transmit a risk signal to a battery management system.

According to the present disclosure, desirably, the cell charging/discharging switch may turn off the charging or discharging operation for the corresponding battery cell if the state of the corresponding battery cell is in the unstable state and turn on the charging or discharging operation for the corresponding battery cell if the unstable state of the corresponding battery cell is changed to the stable state.

To accomplish the above-mentioned objects, according to another aspect of the present disclosure, there is provided a thermal runaway detection and control method of a thermal runaway detection and control device, the method including the steps of: detecting and monitoring hydrogen concentrations in gases of battery cells using palladium-based nano sensors attached to the battery cells; determining whether the detected hydrogen concentrations are over a first reference value; if the detected hydrogen concentrations are over the first reference value, turning off charging/discharging switches of the corresponding battery cells; determining whether the detected hydrogen concentrations are over a second reference value higher than the first reference value; and if the detected hydrogen concentrations are over the second reference value, transmitting a risk signal to a battery management system.

According to the present disclosure, desirably, the palladium-based nano sensor may include at least one of a palladium-based nanogap sensor and a palladium-based nanorod sensor, and the palladium-based nanogap sensor and the palladium-based nanorod sensor may be switched from an off state to an on state according to the hydrogen concentration detected.

According to the present disclosure, desirably, the palladium-based nano sensor may include a plurality of palladium-based nanogap sensors or palladium-based nanorod sensors having different detection ranges.

According to the present disclosure, desirably, the first reference value may be used in determining whether the corresponding battery cells are in unstable states and the second reference value may be used in determining the corresponding battery cells are in risky states.

According to the present disclosure, desirably, the first reference value may be determined between 20 and 50 ppm, and the second reference value may be determined between 200 and 400 ppm.

According to the present disclosure, desirably, the thermal runaway detection and control method may further include the steps of: determining the states of the corresponding battery cells for a predetermined time after the charging/discharging switches of the corresponding battery cells have been turned off; and if the corresponding battery cells are in the stable states, turning on the charging/discharging switches of the corresponding battery cells.

### BRIEF DESCRIPTION OF THE DRAWINGS

The above and other objects, features and advantages of the present disclosure will be apparent from the following detailed description of the preferred embodiments of the disclosure in conjunction with the accompanying drawings, in which:

FIG. 1 is a flowchart showing a process of causing thermal runaway in a lithium-ion battery;

FIG. 2 is a block diagram showing a thermal runaway detection and control device according to the present disclosure;

FIGS. 3A and 3B are graphs showing the concentrations of off-gases produced upon thermal runaway according to the present disclosure;

FIG. 4 is a flowchart showing a thermal runaway detection and control method according to the present disclosure;

FIG. 5 is a flowchart showing a method for controlling an unstable battery cell according to the present disclosure.

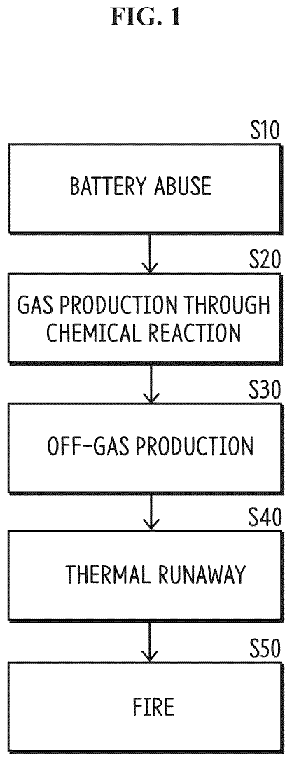

### DETAILED DESCRIPTION OF THE PREFERRED EMBODIMENTS

Hereinafter, the present disclosure is explained with reference to the attached drawings. The present disclosure may be embodied in different forms and should not be construed as limited to the embodiments set forth herein. Further, the corresponding parts in the embodiments of the present disclosure are indicated by corresponding reference numerals and the repeated explanation on the corresponding parts will be avoided.

When it is said that one element is described as being “connected” or “coupled” to the other element, one element may be directly connected or coupled to the other element, but it should be understood that another element may be present between the two elements. In contrast, when it is said that one element is described as being “directly connected” or “directly coupled” to the other element, it should be understood that another element is not present between the two elements.

Terms used in this application are used to only describe specific exemplary embodiments and are not intended to restrict the present disclosure. An expression referencing a singular value additionally refers to a corresponding expression of the plural number, unless explicitly limited otherwise by the context. In this application, terms, such as “comprise”, “include”, or ‘have”, are intended to designate those characteristics, numbers, steps, operations, elements, or parts which are described in the specification, or any combination of them that exist, and it should be understood that they do not preclude the possibility of the existence or possible addition of one or more additional characteristics, numbers, steps, operations, elements, or parts, or combinations thereof.

Hereinafter, an embodiment of the present disclosure will be explained in detail with reference to the attached drawings.

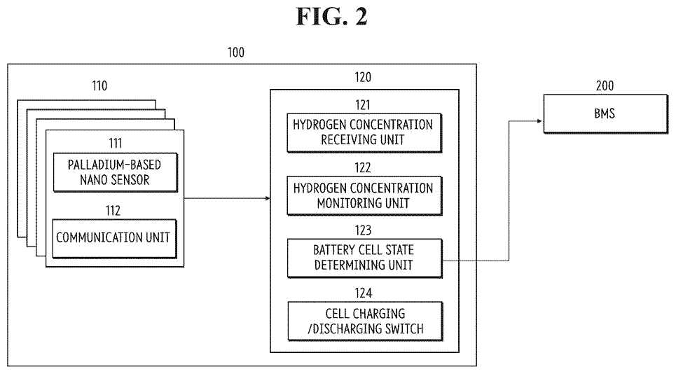

FIG. 2 is a block diagram showing a thermal runaway detection and control device according to the present disclosure.

As shown in FIG. 2, a thermal runaway detection and control device 100 according to the present disclosure includes sensor modules 110 and a battery cell controller 120.

First, the sensor modules 110 are sensors for detecting hydrogen gas produced upon thermal runaway, which are located on a plurality of battery cells.

In the conventional ESS, one gas sensor is mounted on a battery rack or container and performs communication with a battery control system through wired communication, so that there is a limitation in detecting gases generated from individual battery cells. Like this, the conventional technologies are restricted in locating gas sensors on individual battery cells, thereby making it hard to quickly detect thermal runaway and effectively control the battery cells causing troubles.

To solve such problems, the sensor modules 110 according to the present disclosure are provided in the form of thin films and thus attached in the form of magnets or patches to the insides or outsides of the respective battery cells.

Further, each sensor module 110 includes a palladium-based nano sensor 111 and a communication unit 112.

In this case, the palladium-based nano sensor 111 includes a palladium-based nanogap sensor or palladium-based nanorod sensor.

The palladium-based nanogap sensor is a sensor using the expansion of palladium in the case where cracks occurring on palladium formed on a flexible substrate (made of polydimethylsiloxane (PDMS)) of a thin film are exposed to hydrogen gas. The palladium exposed to the hydrogen gas is filled in nanogaps and thus conductive, so that the sensor is switched from an off state to an on state. The palladium-based nanogap sensor detects a hydrogen gas concentration of 100 ppm and is kept at the off state in normal times, thereby operating at low power.

The palladium-based nanorod sensor is made by depositing a palladium layer on a nanorod (e.g., tin dioxide) formed to a height of 200 nm between source drains of an oxide semiconductor. If the palladium layer is exposed to hydrogen (H₂), the hydrogen (H₂) chemically reacts with oxygen ion (O₂) absorbed onto the surface of tin oxide rod (SnO₂) by means of the palladium (Pd) deposited onto the nanorod, thereby decreasing depletion layers of the nanorod. As a result, the resistance value of the nanorod decreases. As the resistance value decreases, a change in source-drain current may be detected, or the source-drain may be turned on and switched from an off state to an on state.

The palladium-based nanorod sensor detects a low hydrogen concentration of 10 ppm and is kept at the off state in normal times, thereby operating at low power.

The palladium-based nano sensor 111 includes a plurality of nanogap sensors or nanorod sensors having different ranges of hydrogen concentrations detected. Otherwise, the palladium-based nano sensor 111 is a combination of nanogap sensors and nanorod sensors.

The communication unit 112 transmits the detected information of the palladium-based nano sensor 111 to the battery cell controller 120 as will be discussed later.

In this case, the communication unit 112 performs wireless communication such as Bluetooth Low Energy (BLE) or ZigBee. The BLE and ZigBee operate at low power so that they can be used for long hours by means of one time battery charging.

When the palladium-based nano sensor 111 is switched from an off state to an on state, the communication unit 112 only transmits the detected information of the palladium-based nano sensor 111, and therefore, it is possible to make use of another low-power wireless communication protocol capable of only transmitting the detected information.

Accordingly, the sensor module 110 is a kind of dry contact sensor and thus has power consumption for a long period of time in the unit of μA as a power level in a battery sleep mode.

The battery cell controller 120 monitors a preceding phenomenon of the thermal runaway of the battery cells, based on the hydrogen concentration information detected by the sensor modules 110 and thus controls charging/discharging switches of the battery cells. In this case, the battery cells as control objects of the battery cell controller 120 are minimal units of the battery components controllable according to the detected results of the sensor modules 110.

As shown in FIG. 2, the battery cell controller 120 includes a hydrogen concentration receiving unit 121, a hydrogen concentration monitoring unit 122, a battery cell state determining unit 123, and a cell charging/discharging switch 124.

The hydrogen concentration receiving unit 121 receives the hydrogen concentration information from the sensor modules 110. According to the embodiment of the present disclosure, the hydrogen concentration receiving unit 121 receives hydrogen concentrations of at least 20 ppm or more.

If hydrogen emission is detected from the hydrogen concentration information, the hydrogen concentration monitoring unit 122 monitors in real time the hydrogen concentration information received in the hydrogen concentration receiving unit 121. For example, the hydrogen concentration monitoring unit 122 monitors whether the hydrogen concentration increases up to a first reference value within a predetermined time after the hydrogen emission has been detected. If the hydrogen concentration increases up to the first reference value, the hydrogen concentration monitoring unit 122 monitors whether the hydrogen concentration increases up to a second reference value within a predetermined time.

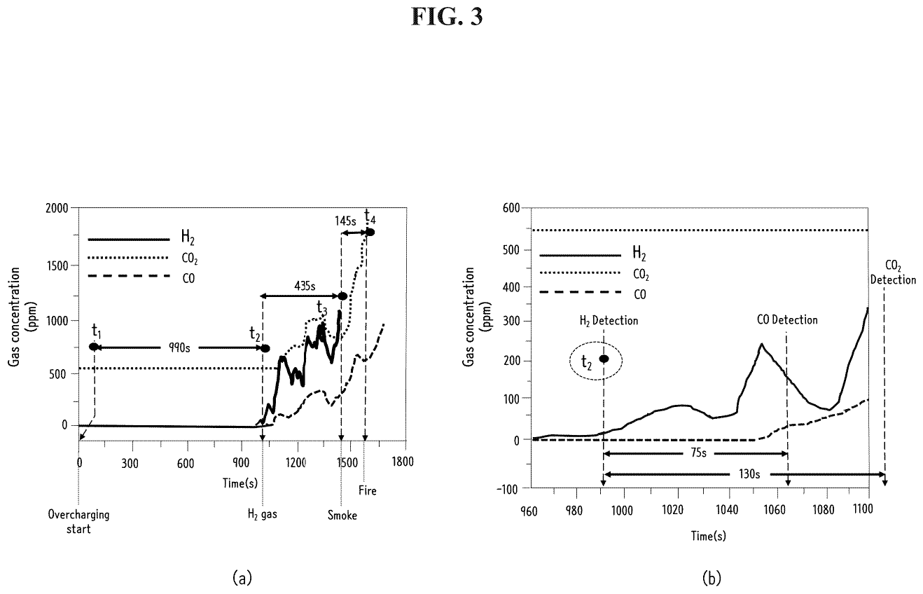

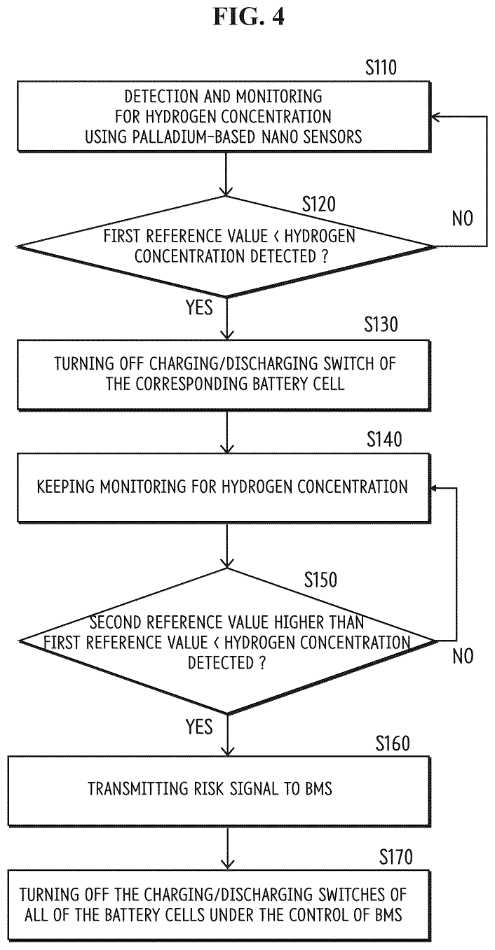

In this case, the real-time hydrogen concentration monitoring operation of the hydrogen concentration monitoring unit 122 will be explained in detail with reference to FIGS. 3a to 4 later.

The battery cell state determining unit 123 determines the states of the battery cells, based on the results monitored in real time through the hydrogen concentration monitoring unit 122.

In this case, the battery cell state determining unit 123 determines the stable or unstable states of the individual battery cells corresponding to the sensor modules 110 according to the hydrogen concentrations monitored by the sensor modules 110. For example, the battery cell state determining unit 123 determines the states of the battery cells as stable, unstable, and risky states according to non-detection of hydrogen, the hydrogen concentration reaching the first reference value, and the hydrogen concentration reaching the second reference value.

For example, if the hydrogen concentration reaches the first reference value, the battery cell, which corresponds to the sensor module 110 measuring the hydrogen concentration reaching the first reference value, is determined as that in the unstable state.

Further, if the hydrogen concentration reaches the second reference value, the battery cell, which corresponds to the sensor module 110 measuring the hydrogen concentration reaching the second reference value is determined as that in the risky state, and therefore, the battery cell state determining unit 123 transmits information, that is, a risk signal to a battery management system (BMS) 200 for an ESS or battery pack, so that the BMS 200 turns off the charging/discharging switches of all of the battery cells. If the risk signal (abnormal signal) for the battery cell is received, the BMS 200 performs battery power shutoff and temperature control and controls operations of a thermal runaway control device having a fire extinguishing controller. Even if not shown in the drawings, an alarm is generated and current situation information is transmitted to a control center under the control of the BMS 200, thereby preparing such an emergency situation.

The cell charging/discharging switch 124 performs cell charging/discharging switching according to the determined results of the battery cell state determining unit 123. For example, if it is determined that the hydrogen concentration reaches the first reference value to determine the corresponding battery cell as the battery cell in the unstable state, the cell charging/discharging switch 124 turns off the charging or discharging operation for the corresponding battery cell. After that, if the unstable state of the corresponding battery cell is changed to the stable state, the cell charging/discharging switch 124 turns on the charging or discharging operation for the corresponding battery cell.

According to the embodiment of the present disclosure, if the first reference value is set to a low hydrogen concentration of 20 ppm, the sensor modules 110 may detect hydrogen due to disturbance or noise in the air, and therefore, if the hydrogen concentration decreases as time passes, it is possible that the corresponding battery cell returns to the stable state.

According to the embodiment of the present disclosure, like this, a low hydrogen concentration is quickly detected, and charging or discharging only for individual battery cells through the monitoring is turned on and off, thereby efficiently controlling thermal runaway, without having any shutdown for the charging or discharging for the entire battery.

Hereinafter, hydrogen concentrations varied according to time at the initial step of thermal runaway will be explained with reference to FIGS. 3A and 3B.

FIGS. 3A and 3B are graphs showing the concentrations of off-gases produced upon thermal runaway according to the present disclosure.

In detail, FIGS. 3A and 3B are thermal runaway monitoring test result graphs showing the concentrations of off-gases produced as time passes.

As shown in FIG. 3A, if thermal runaway occurs, carbon dioxide (CO₂), hydrogen (H₂), and carbon monoxide (CO) increase in concentration.

First, a battery cell starts to be overcharged at a time point of t1, as shown in FIG. 3A, and off-gases are produced at a time point of t2, that is, at 990 seconds from the t1. If the off-gases are produced, as shown in FIG. 3A, it is checked that hydrogen (H₂), carbon dioxide (CO₂), and carbon monoxide (CO) are produced, while having different concentrations and increasing rates. At a time point t3 of 1425 seconds after 435 seconds have passed from the production of the off-gases (from the time point of t2), secondary venting in which smoke is generated is caused.

FIG. 3B is a graph enlarging the initial section of the off-gases produced in FIG. 3A. Referring to FIG. 3B, it is appreciated that hydrogen (H₂) is first produced at the t2 and amounts of carbon dioxide (CO₂) and carbon monoxide (CO) produced are remarkably smaller than an amount of hydrogen (H₂) produced. For example, carbon monoxide (CO) having a concentration of 50 ppm or less is sensed in 75 seconds after the t2 where the off-gases are produced.

As a result, it is checked that the concentration of hydrogen (H₂) is higher than the concentrations of other gases within the initial short time of thermal runaway, and to early prevent thermal runaway from occurring, therefore, it is optimal that changes in hydrogen concentration are monitored and controlled according to monitored results.

As mentioned above, hydrogen (H₂) is sensed in the initial step of the production of off-gases, thereby detecting thermal runaway most early. Hydrogen (H₂) among the off-gases is most early detected, but an absolute value of hydrogen concentration is lower than 100 ppm in the initial step. Even in 130 seconds from the production of the off-gases, the absolute value of hydrogen concentration is lower than 400 ppm.

To detect hydrogen (H₂) most early produced upon thermal runway, therefore, it is necessary to adopt a sensor having high sensitivity capable of detecting a low hydrogen concentration.

According to the embodiment of the present disclosure, the palladium-based nano sensor 111 detects the hydrogen concentration between 20 and 200 ppm, and after detecting, it monitors the hydrogen concentration for 130 seconds.

During the monitoring section, the temperature of the battery cell is kept to a temperature between 35 and 56° C., and therefore, the palladium-based nano sensor 111 is not damaged by heat and detects the hydrogen concentration accurately.

To detect the hydrogen concentration between 20 and 200 ppm, the palladium-based nano sensor 111 is made of a combination of a sensor for detecting 10 ppm concentration and a sensor for detecting 100 ppm concentration.

For example, the palladium-based nanorod sensor is adopted to detect hydrogen concentration of 10 ppm, and the palladium-based nanogap sensor is adopted to detect hydrogen concentration of 100 ppm.

FIG. 4 is a flowchart showing a thermal runaway detection and control method according to the present disclosure.

As shown in FIG. 4, the thermal runaway detection and control device detects and controls the thermal runaway for the battery cells from the hydrogen concentration.

At step S110, the thermal runaway detection and control device detects hydrogen produced using the palladium-based nano sensors attached to the respective battery cells and monitors hydrogen concentrations of the battery cells. According to the embodiment of the present disclosure, if the hydrogen concentration greater than or equal to 20 ppm is detected, the monitoring operation for the hydrogen concentration starts to be performed.

At step S120, the thermal runaway detection and control device compares the hydrogen concentration values of the battery cells for a predetermined time with a first reference value, while monitoring the hydrogen concentrations.

If the detected hydrogen concentration values are not over the first reference value (e.g., 50 ppm), the thermal runaway detection and control device keeps monitoring for hydrogen concentrations.

Contrarily, if the detected hydrogen concentration value is over the first reference value (e.g., 50 ppm), at step S130, the thermal runaway detection and control device selectively turns off the charging/discharging switch of the corresponding battery cell having the hydrogen concentration over the first reference value.

If the charging/discharging switch of the corresponding battery cell is turned off, an electrical operation of the corresponding battery cell is not performed to early prevent the thermal runaway of the corresponding battery cell from occurring. That is, the thermal runaway detection and control device does not turn off the charging/discharging switches of all of the battery cells, but turns off only the charging/discharging switch of the corresponding battery cell, thereby improving the efficiency in the use of the battery.

At step S140, the thermal runaway detection and control device keeps monitoring for hydrogen concentration after the charging/discharging switch of the corresponding battery cell has been turned off.

Even after the charging/discharging switch of the corresponding battery cell has been turned off due to the hydrogen concentration value over the first reference value, this is because hydrogen is detected from the off-gases if the corresponding battery cell does not have any thermal runaway or the factors affecting the thermal runaway are not removed even through the off operation of the switch.

At step S150, the thermal runaway detection and control device monitors whether the hydrogen concentration reaches a second reference value higher than the first reference value.

If the detected hydrogen concentration value is not over the second reference value (e.g., 200 ppm), the thermal runaway detection and control device keeps monitoring for hydrogen concentrations.

Contrarily, if the detected hydrogen concentration value is over the second reference value (e.g., 200 ppm), at step S160, the thermal runaway detection and control device transmits the risk signal to the BMS, thereby allowing all of the battery cells of the ESS or battery pack to be controlled.

At step S170, the thermal runaway detection and control device turns off the charging/discharging switches of all of the battery cells of the ESS or battery pack under the control of the BMS. Further, the thermal runaway detection and control device transmits the risk signal to an operator through the BMS.

In controlling all of the battery cells through the BMS, the time during which the hydrogen concentration reaches the second reference value is considered. For example, if the hydrogen concentration value reaches the second reference value (e.g., 200 ppm) within a time between 75 and 130 seconds during which the hydrogen concentration increases in the process of producing off-gases.

In detail, the second reference value of 200 ppm is a substantially low hydrogen concentration with which it is hard to detect thermal runaway, but as shown in FIG. 3, if the hydrogen concentration value reaches the second reference value within a time between 75 and 130 seconds after the production of hydrogen has been detected, it is determined that the possibility of off-gas leakage is high.

According to the embodiment of the present disclosure, a small amount of hydrogen is monitored step by step according to time to effectively detect the preceding phenomenon of thermal runaway, thereby preventing the thermal runaway from occurring and minimizing the damage caused by the thermal runaway.

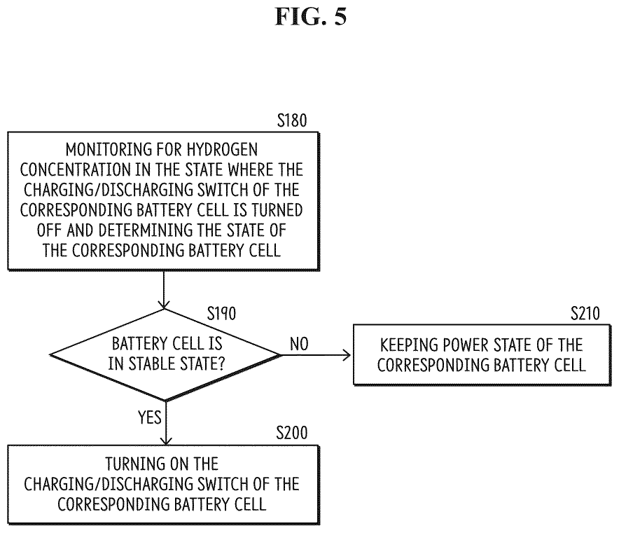

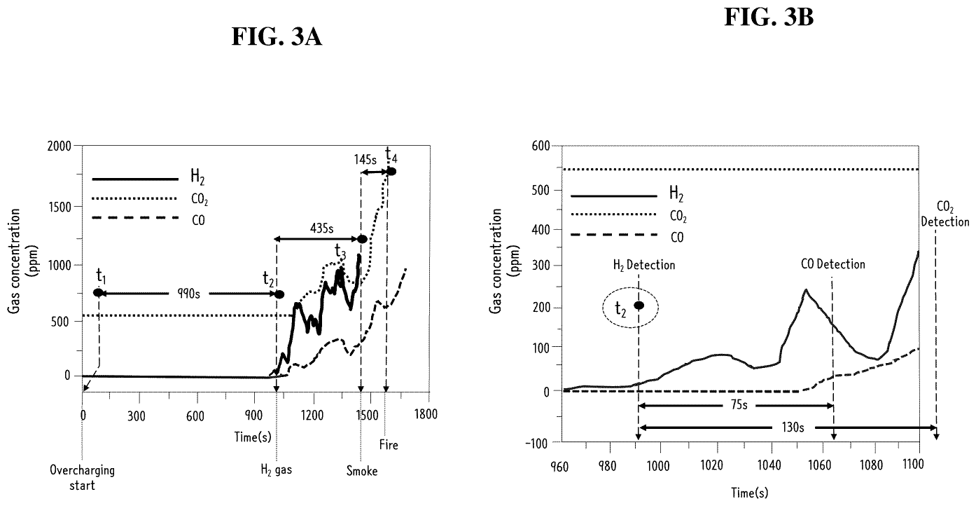

FIG. 5 is a flowchart showing a method for controlling an unstable battery cell according to the present disclosure.

As explained with reference to FIG. 4, if the detected hydrogen concentration value is over the first reference value (e.g., 50 ppm), the thermal runaway detection and control device selectively turns off the charging/discharging switch of the corresponding battery cell having the hydrogen concentration over the first reference value.

At step S180, the hydrogen concentration of the corresponding battery cell is monitored to determine the state of the battery cell after the charging/discharging switch of the corresponding battery cell has been turned off. Since the first reference value is a substantially low hydrogen concentration, there is a possibility that hydrogen is not generated as off-gas, but detected from disturbance or noise.

At step S190, therefore, the stable state of the corresponding battery cell is determined according to the variations in the hydrogen concentration of the battery cell turned off after a given time has passed. If the hydrogen concentration decreases to cause the corresponding battery cell to be in a stable state, at step S200, the charging/discharging switch of the corresponding battery cell is turned on.

Contrarily, if the corresponding battery cell is not in the stable state, at step S210, the charging/discharging switch of the corresponding battery cell is kept at the current state (turned off). The thermal runaway detection and control device returns to the step S140 of FIG. 4 after the step S210 has been performed and keeps hydrogen concentration monitoring.

According to the embodiment of the present disclosure, the thermal runaway detection and control device turns off the switch of the corresponding battery cell having a possibility of thermal runaway, checks the state of the corresponding battery cell after a given time has passed, and converts the switch turned off into the switch turned on according to the checked state of the battery cell, thereby improving the efficiency in the use of the battery cell.

As described above, the thermal runaway detection and control device and method according to the present disclosure can accurately detect a small amount of hydrogen produced at the extremely initial step of thermal runaway using the palladium-based nano sensors mounted on the individual battery cells of the lithium-ion battery.

Further, the thermal runaway detection and control device and method according to the present disclosure monitors the hydrogen concentrations detected, controls the charging/discharging switches of the individual battery cells according to the monitored hydrogen concentrations, and pre-sensing and preventing the thermal runaway of the lithium-ion battery.

Furthermore, the thermal runaway detection and control device and method according to the present disclosure controls the charging/discharging switches according to the stable states of the battery cells monitored according to the variations in the hydrogen concentrations detected, thereby preventing thermal runaway from occurring and efficiently controlling the use of the battery cells.

The disclosed embodiments are merely exemplary of the disclosure, which can be embodied in various forms. Therefore, specific structural and functional details disclosed herein are not to be interpreted as limiting, but merely as a basis for the claims and as a representative basis for teaching one of ordinary skill in the art to variously employ the present disclosure in virtually any appropriately detailed structure. For example, the parts expressed in a singular form may be dispersedly provided, and in the same manner as above, the parts dispersed may be combined with each other.

Accordingly, the scope of the present disclosure is limited by the claims appended hereto, and various embodiments and modifications can be made without departing from the scope of the claims below.

What is claimed is:

1. A thermal runaway of lithium-ion battery detection and control device comprising:
sensor modules for detecting and monitoring hydrogen concentrations of battery cells and each having a palladium-based nano sensor and a communication unit; and
a battery cell controller for turning on and off charging/discharging switches of the battery cells according to the hydrogen concentrations detected through the sensor modules and variations in the hydrogen concentrations according to time,
wherein the palladium-based nano sensor detects the hydrogen concentration between 20 and 400 ppm.

2. The thermal runaway of lithium-ion battery detection and control device according to claim 1, wherein the palladium-based nano sensor comprises at least one of a palladium-based nanogap sensor and a palladium-based nanorod sensor, and the palladium-based nanogap sensor and the palladium-based nanorod sensor are switched from an off state to an on state according to the hydrogen concentration detected.

3. The thermal runaway of lithium-ion battery detection and control device according to claim 2, wherein the palladium-based nano sensor comprises a plurality of palladium-based nanogap sensors or palladium-based nanorod sensors having different detection ranges.

4. The thermal runaway of lithium-ion battery detection and control device according to claim 2, wherein the battery cell controller comprises:
a hydrogen concentration receiving unit for receiving the hydrogen concentrations from the sensor modules;
a hydrogen concentration monitoring unit for monitoring the hydrogen concentrations in real time;
a battery cell state determining unit for determining the states of the battery cells, based on the results monitored in real time through the hydrogen concentration monitoring unit; and
a cell charging/discharging switch for turning on and off the charging/discharging switches of the battery cells according to the determined results of the battery cell state determining unit.

5. The thermal runaway of lithium-ion battery detection and control device according to claim 4, wherein the hydrogen concentration monitoring unit monitors a first reference value in the hydrogen concentration through which it is determined that the corresponding battery cell is in an unstable state and a second reference value in the hydrogen concentration through which it is determined that the corresponding battery cell is in a risky state.

6. The thermal runaway of lithium-ion battery detection and control device according to claim 5, wherein the first reference value is determined between 20 and 50 ppm, and the second reference value is determined between 200 and 400 ppm.

7. The thermal runaway of lithium-ion battery detection and control device according to claim 5, wherein the battery cell state determining unit determines the states of the battery cells as stable, unstable, and risky states according to non-detection of hydrogen, the hydrogen concentration reaching the first reference value, and the hydrogen concentration reaching the second reference value.

8. The thermal runaway of lithium-ion battery detection and control device according to claim 5, wherein if the hydrogen concentration of the corresponding battery cell reaches the second reference value 75 seconds before from the detection of hydrogen production, the battery cell state determining unit determines the state of the corresponding battery cell as the risky state and transmits a risk signal to a battery management system.

9. The thermal runaway of lithium-ion battery detection and control device according to claim 5, wherein the cell charging/discharging switch turns off the charging or discharging operation for the corresponding battery cell if the state of the corresponding battery cell is in the unstable state and turns on the charging or discharging operation for the corresponding battery cell if the unstable state of the corresponding battery cell is changed to the stable state.

10. A thermal runaway of lithium-ion battery detection and control method of a thermal runaway detection and control device, the method comprising the steps of:
detecting and monitoring hydrogen concentrations in gases of battery cells using palladium-based nano sensors attached to the battery cells;
determining whether the detected hydrogen concentrations are over a first reference value;
if the detected hydrogen concentrations are over the first reference value, turning off charging/discharging switches of the corresponding battery cells;
determining whether the detected hydrogen concentrations are over a second reference value higher than the first reference value; and
if the detected hydrogen concentrations are over the second reference value, transmitting a risk signal to a battery management system.

11. The thermal runaway of lithium-ion battery detection and control method according to claim 10, wherein the palladium-based nano sensor comprises at least one of a palladium-based nanogap sensor and a palladium-based nanorod sensor, and the palladium-based nanogap sensor and the palladium-based nanorod sensor are switched from an off state to an on state according to the hydrogen concentration detected.

12. The thermal runaway of lithium-ion battery detection and control method according to claim 11, wherein the palladium-based nano sensor comprises a plurality of palladium-based nanogap sensors or palladium-based nanorod sensors having different detection ranges.

13. The thermal runaway of lithium-ion battery detection and control method according to claim 10, wherein the first reference value is used in determining whether the corresponding battery cells are in unstable states and the second reference value is used in determining the corresponding battery cells are in risky states.

14. The thermal runaway of lithium-ion battery detection and control method according to claim 13, wherein the first reference value is determined between 20 and 50 ppm, and the second reference value is determined between 200 and 400 ppm.

15. The thermal runaway of lithium-ion battery detection and control method according to claim 13, further comprising the steps of:
determining the states of the corresponding battery cells for a predetermined time after the charging/discharging switches of the corresponding battery cells have been turned off; and
if the corresponding battery cells are in the stable states, turning on the charging/discharging switches of the corresponding battery cells.
